(19) United States
(12) Patent Application Publication
(10) Pub. No.: US 2026/0368901 A1
(43) Pub. Date: Nov. 19, 2026

(54) SIMULTANEOUS RECONSTRUCTION AND SEGMENTATION FOR SMALL STRUCTURES

(71) Applicant: Canon Kabushiki Kaisha, Tokyo (JP)

(72) Inventor: Paul THOMSON, Edinburgh (GB)

(73) Assignee: Canon Kabushiki Kaisha, Tokyo (JP)

(21) Appl. No.: 19/206,349

(22) Filed: May 13, 2025

(51) Int. Cl. G06T 7/12; A61B 6/00; A61B 6/03; A61B 6/42; G06T 12/20

(52) U.S. Cl. CPC G06T 7/12; A61B 6/032; A61B 6/5205; G06T 12/20; A61B 6/4241; A61B 6/5258; G06T 2207/10072; G06T 2207/10116; G06T 2207/20081; G06T 2207/30004; G06T 2210/41; G06T 2211/441

(57) ABSTRACT

According to embodiments, a reconstructed image is obtained based on the plurality of X-ray measurements and then processed to obtain segmentations of a number of structures in the image. A differentiable simulation is applied to the segmentations to produce a simulated sinogram. The differentiable simulation may be trained to model the physical processes and the way in which different tissue types, as identified by segmentations of the image, interact with the radiation. The simulated sinogram represents the expected X-ray measurements (i.e. the forward projections) based upon the reconstructed image and the segmentation. To evaluate the accuracy of the segmentations, the simulated sinogram is compared to a sinogram representing the actual X-ray measurements and a loss measurement is obtained. This loss measurement is used to update the segmentations to produce more accurate segmentations.

### TECHNICAL FIELD

The present disclosure relates to a data processing system and a method for processing X-ray measurements and in particular for processing the X-ray measurements to obtain segmentations of structures of a subject.

### BACKGROUND

Computed tomography is an imaging technique using a plurality of X-ray measurements obtained at different angles to reconstruct an internal image of the subject. Different techniques may be used to reconstruct the image based on the plurality of X-ray measurements, including filtered back-projection or iterative reconstruction. The problem of reconstruction in some circumstances is well-posed, in which case the X-ray measurements are sufficient for performing accurate reconstruction of the images of the subject. Alternatively, the problem of reconstruction may be ill-posed, in which case artefacts may be present in the reconstructed images. The reconstruction problem may be ill-posed due to X-ray measurements being collected over only a small range of angles, due to only a small number of X-ray measurements being collected or due to a source of noise in the measurements.

When reconstructed images are produced from the X-ray measurements, these may be processed by applying a segmentation algorithm in order to identify and represent distinct structures in the image. For example, the image may be processed to represent in the image, different organs, such as the heart or lungs, of a subject or to identify different bones of the subject. When the reconstruction problem is well-posed and the image of the subject may be accurately reconstructed, the segmentation of that image may be reliably achieved. However, if the reconstructed images contain artefacts, the result of applying a segmentation algorithm to these images may result in inaccurate segmentation of the structures of the subject.

### BRIEF DESCRIPTION OF DRAWINGS

Some embodiments of the disclosure will now be described, by way of example only and with reference to the accompanying drawings, in which:

FIG. 1 illustrates a schematic of a computing device according to example embodiments;

FIG. 2 illustrates a schematic of a computing system according to example embodiments;

FIG. 3 illustrates a schematic of an X-ray imaging system;

FIG. 4 illustrates a process for obtaining segmentations of the internal structure of a subject based on a plurality of X-ray projections obtained using the X-ray imaging system;

FIG. 5 illustrates the production of separate segmentation meshes based on voxels of image data;

FIG. 6 illustrates a sinogram and sinogram data captured at multiple different projection angles;

FIG. 7 illustrates how the differentiable simulation may be used to reconstruct sinogram data based segmentation data representing segmentations at different times;

FIG. 8 illustrates a simplified schematic of a neural network;

FIGS. 9A and 9B illustrate simplified examples of generative adversarial neural networks that may be used to update the segmentations;

FIG. 10 illustrates an example of a diffusion network for updating the segmentations; and

FIG. 11 illustrates a method according to example embodiments.

### DETAILED DESCRIPTION

It is desirable to improve the accuracy of the segmentation of structures present in an image, in particular in the case that the reconstruction problem is ill-posed.

According to a first aspect, there is provided a data processing system comprising processing circuitry configured to: obtain a plurality of X-ray measurements of a subject obtained at different angles of an X-ray source and X-ray detector used to provide the X-ray measurements; process the X-ray measurements to produce a sinogram; perform reconstruction using the plurality of X-ray measurements to produce at least one image of the subject; apply a segmentation algorithm to the at least one image to obtain segmentations based on different structures within the at least one image; apply a simulation to the segmentations to produce a simulated sinogram representing forward projections predicted based on the segmentations; and compute a loss metric based on a comparison between the sinogram and the simulated sinogram; and update the segmentations based on the loss metric.

According to embodiments, a reconstructed image is obtained based on the plurality of X-ray measurements and then processed to obtain segmentations of a number of structures in the image. A differentiable simulation is applied to the segmentations to produce a simulated sinogram. Furthermore, additional parameters may also be provided as inputs to the simulation. These additional parameters may comprise parameters indicating one or more of tissue properties, scanner geometry information, scan protocol. The simulation may be trained or constructed to model the physical processes and the way in which different tissue types, as identified by segmentations of the image, interact with the radiation. The simulated sinogram represents the expected X-ray measurements (i.e. the forward projections) based upon the reconstructed image and the segmentation. To evaluate the accuracy of the segmentations, the simulated sinogram is compared to a sinogram representing the actual X-ray measurements and a loss measurement is obtained. This loss measurement is used to update the segmentations to produce more accurate segmentations.

According to a second aspect, there is provided a method comprising: obtaining a plurality of X-ray measurements of a subject obtained at different angles of an X-ray source and X-ray detector used to provide the X-ray measurements; processing the X-ray measurements to produce a sinogram; performing reconstruction using the plurality of X-ray measurements to produce at least one image of the subject; applying a segmentation algorithm to the at least one image to obtain segmentations based on different structures within the at least one image; applying a simulation to the segmentations to produce a simulated sinogram representing forward projections predicted based on the segmentations; and computing a loss metric based on a comparison between the sinogram and the simulated sinogram; and updating the segmentations based on the loss metric.

According to third aspect, there is provided a computer program comprising one or more sets of computer readable instructions, which when executed by at least one processor, cause a method to be performed, the method comprising: obtaining a plurality of X-ray measurements of a subject obtained at different angles of an X-ray source and X-ray detector used to provide the X-ray measurements; processing the X-ray measurements to produce a sinogram; performing reconstruction using the plurality of X-ray measurements to produce at least one image of the subject; applying a segmentation algorithm to the at least one image to obtain segmentations based on different structures within the at least one image; applying a simulation to the segmentations to produce a simulated sinogram representing forward projections predicted based on the segmentations; and computing a loss metric based on a comparison between the sinogram and the simulated sinogram; and updating the segmentations based on the loss metric.

According to fourth aspect, there is provided a non-transitory computer readable medium storing a computer program comprising one or more sets of computer readable instructions, which when executed by at least one processor, cause a method to be performed, the method comprising: obtaining a plurality of X-ray measurements of a subject obtained at different angles of an X-ray source and X-ray detector used to provide the X-ray measurements; processing the X-ray measurements to produce a sinogram; performing reconstruction using the plurality of X-ray measurements to produce at least one image of the subject; applying a segmentation algorithm to the at least one image to obtain segmentations based on different structures within the at least one image; applying a simulation to the segmentations to produce a simulated sinogram representing forward projections predicted based on the segmentations; and computing a loss metric based on a comparison between the sinogram and the simulated sinogram; and updating the segmentations based on the loss metric.

Embodiments will be described in more detail with reference to the accompanying Figures.

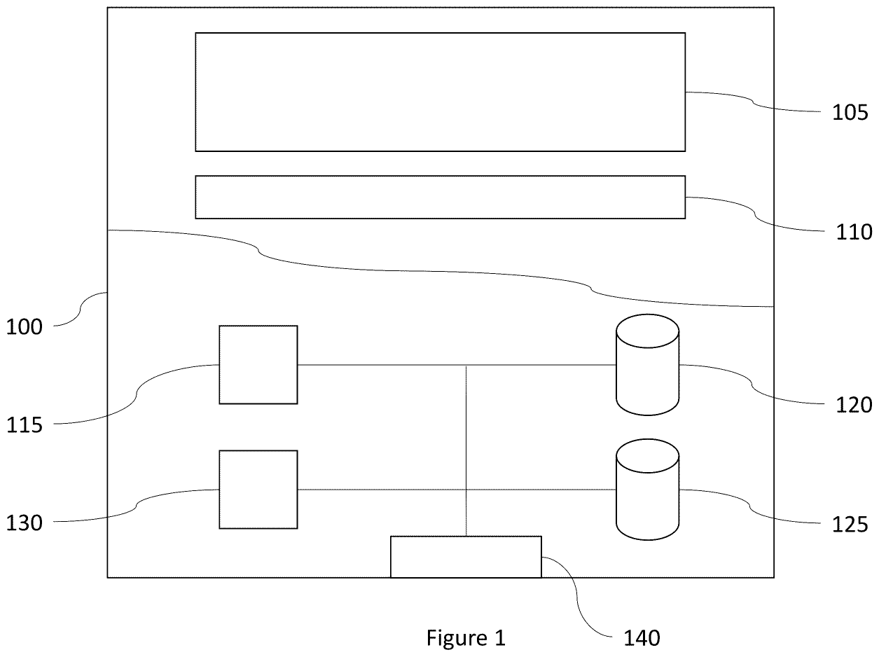

Reference is made to FIG. 1, which illustrates an example data processing system 100 in which embodiments may be implemented. The system 100 may be a server, a terminal or workstation, a personal computer (PC), or some other form of device.

The system 100 may comprise an interface 140 over which it sends and receives signals. The interface 140 may be a wired or wireless interface. For instance, the interface 140 may comprise a wired interface for connection to a wired network (e.g. a local area network and/or the internet). Alternatively or in addition, the interface 140 may comprise transceiver apparatus configured to send and receive communications over a radio interface. The transceiver apparatus may be provided, for example, by means of a radio part and associated antenna arrangement. The antenna arrangement may be arranged internally or externally to the system 100.

The system 100 is provided with at least one data processing entity 115, at least one random access memory 120, at least one read only memory 125, and other possible components 130 for use in software and hardware aided execution of tasks it is designed to perform, including control of, access to, and communications with access systems and other communication devices. The at least one random access memory 120 and the hard drive 125 are in communication with the data processing entity 115, which may be a data processor. The data processing, storage and other relevant control apparatus can be provided on an appropriate circuit board and/or in chipsets. A user may controls the operation of the system 100 by means of a suitable user interface such as key pad 110, or by voice commands. A display 105 may be included on the system 100 for displaying visual content to a user. The system 100 may also comprise a speaker for providing audio content.

The memory of the system 100 (i.e. the random access memory 120 and the hard drive 125) may be configured to store computer readable instructions for execution by the data processor 115 to perform the data processing functions described herein as being performed by the system 100. Alternatively, the components 130 may comprise hardware components, such as a field programmable gate array (FPGA) or application specific integrated circuit (ASIC), for performing the operations described herein as being performed by the system 100. In some embodiments, the operations described herein as being performed by the system 100 may be performed by a combination of the hardware components 130 or by the processor 115 executing computer readable instructions.

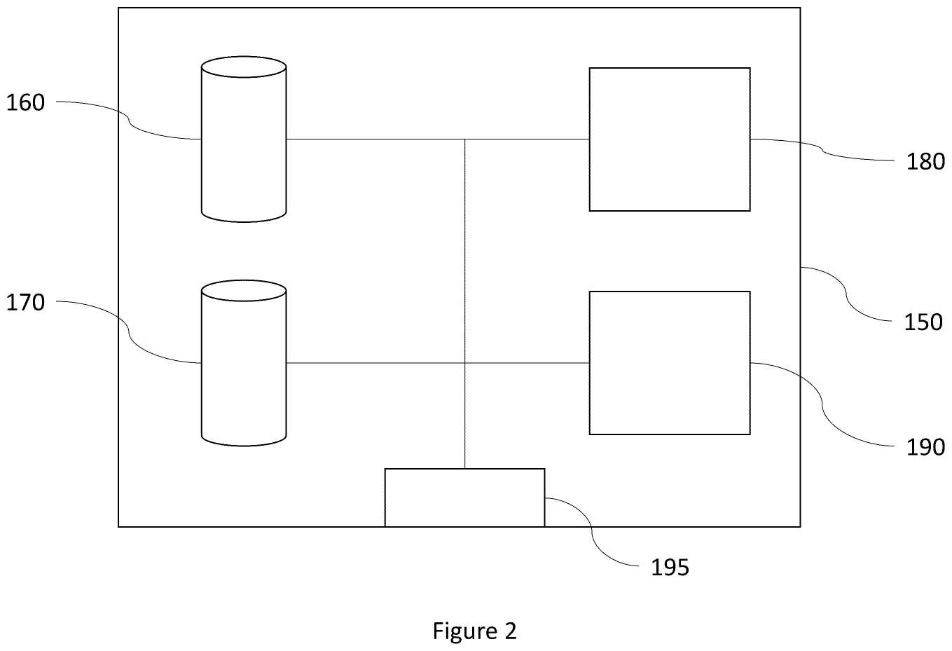

Reference is made to FIG. 2, which illustrates an example computer system 150 that may also be used for performing processing described herein. In particular, the example computer system 150 may be used for performing the training of machine learning models discussed herein. Additionally or alternatively, the computer system 150 may be used to perform other types of processing in conjunction with another computer system, such as the data processing system 100. The system 150 is shown as a single enclosed apparatus. However, in some embodiments, the system 150 is a distributed system, with multiple data processing apparatuses operating in communication with one other. The system 150 may comprise a server, back-end system, or the like.

The system 150 comprises at least one random access memory 160, at least one hard drive 170, at least one data processing unit 180, 190 and an input/output interface 195. The memories 160, 170, store data for processing and instructions for executing by the at least one data processing unit 180, 190. In the case that the system 150 is used for training one or more machine learning models, the memories 160, 170 may store the training data, which is applied to train the machine learning models. The memories 160, 170 may store computer executable code which, when executed by at least one data processing unit 180, 190, provide the one or more models or perform other operations. The at least one data processing unit 180, 190 may perform the training of the models, any necessary pre-processing of data for use by the models. Via the interface 195, the system 150 receives the data items for processing by the at least one data processing unit 180, 190. The system 150 may additionally send via the interface 195, the results produced by the processing performed by the at least one data processing unit 180, 190.

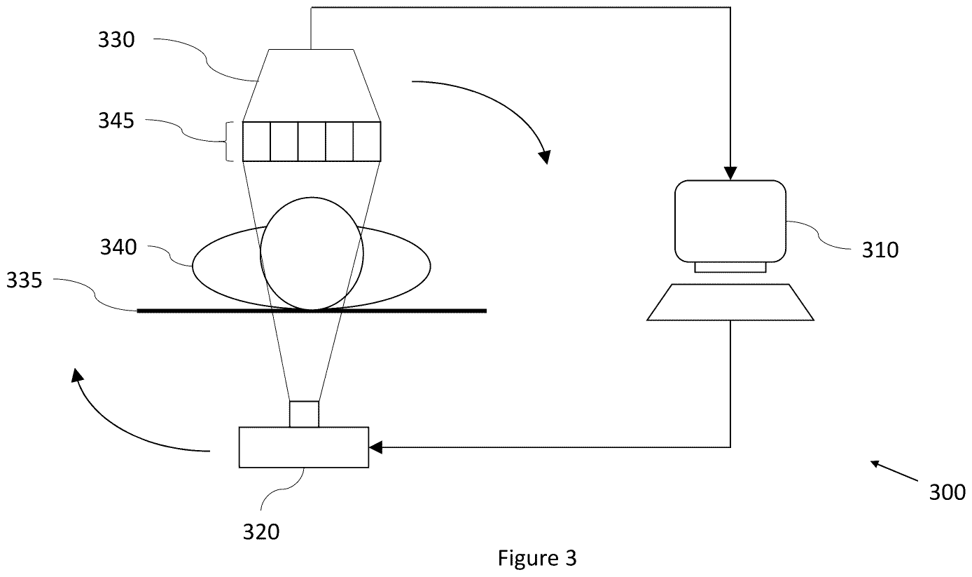

Reference is made to FIG. 3, which illustrates a system 300 comprising an X-ray imaging apparatus and a data processing system 310 connected to the X-ray imaging apparatus. The data processing system 310 may be provided by either or both of the systems 100, 150 described above. Reference herein to operations being performed by the data processing system 310 are understood to refer to operations performed by the processing circuitry (e.g. by a processor executing computer readable instructions) of the data processing system 310.

The X-ray imaging apparatus may be referred to as a computer tomography (CT) scanner. The X-ray imaging apparatus comprises the X-ray tube 320 (which may also be referred to as the X-ray generator 320 or X-ray source 320), and an X-ray detector apparatus 330. Located between the X-ray tube 320 and the X-ray detector apparatus 330 is a subject 340 lying on a table 335. The subject 340 is a patient who is being imaged using the X-ray apparatus.

The X-ray detector apparatus 330 comprises a plurality of detectors 345, which may also be referred to as detector elements 345. Each of the detector elements 345 receives X-rays emitted at a different angle from the X-ray source 320. FIG. 3 shows an example in which the X-rays are emitted from the X-ray source 320 in a fan beam configuration in which the width of the X-ray beam increases with the distance from the source 320. However, other configurations such as a parallel beam configuration are also possible.

Since the X-ray detector apparatus 330 comprises a plurality of detectors 345, when the X-ray source 320 and the X-ray detector apparatus 330 are arranged in a given position, a plurality of X-ray measurements equal to the number of detectors 345 may be made. Each of these X-ray measurements may be referred to as an X-ray projection and provides a measurement of the X-ray attenuation along an associated path through the subject 340. When making these X-ray measurements, the X-ray source 320 emits one or more pulses of X-rays for the given position in which the source 320 and detection apparatus 330 are arranged. Each of the detectors 345 measures the intensity of X-rays it receives when arranged in a given position, where that intensity is inversely related to the level of attenuation along the path from the X-ray source 320 to the detector 345 when the detector is located at that position.

During operation of the X-ray imaging apparatus, the X-ray tube 320 and X-ray detector apparatus 330 may rotate around the subject 340, so as to capture X-ray measurements at different angles. For a given position of the subject 340, the X-ray measurements may be taken through a slice through the subject 340. To capture measurements through additional slices of the subject 340, the table 335 may be operable to move in a direction parallel to the axis of the X-ray imaging apparatus. For example, the table 335 may be provided with a motor configured to move the table 335 in a direction parallel to the axis of the imaging apparatus. By moving the table 335 along the axis of the imaging apparatus, in addition to capturing X-ray measurements at different angles about the subject 340, the measurement data captures three dimensional imaging data related to the subject 340.

The X-ray detectors 345 may, in addition to determining the intensity of received X-rays, also measure the frequency of the received X-ray photons. In this way, the detectors 345 are able to provide a frequency spectrum of the X-ray data they receive. In this case, the X-ray imaging technique employed by the system 300 is referred to as photon-counting computed tomography (PCCT). The advantage of this technique is that the data processing system 310 has additional information to use for producing the reconstructed images and corresponding sinogram from the X-ray measurement data. The information can be further enhanced by injecting a contrast agent into the subject 340, where that contrast agent 340 has different attenuation coefficients for different parts of the frequency spectrum as compared to the surrounding tissue. In this way, the location of the contrast agent in the reconstructed images may be determined based on the frequency spectra of the X-ray measurements.

Therefore, a plurality of X-ray measurements are collected, each of which is associated with a different line (at a different angle and/or through a different slice) passing through the subject 340. The X-ray measurements are provided to the data processing system 310, which is configured to process the X-ray measurements to reconstruct an internal image of the subject 340 and to apply a segmentation algorithm to derive a plurality of segmentations associated with different structures of the subject 340 or a plurality of segmentation meshes. The plurality of segmentations may be collectively referred to herein as segmentation information, segmentation data or a segmented volume.

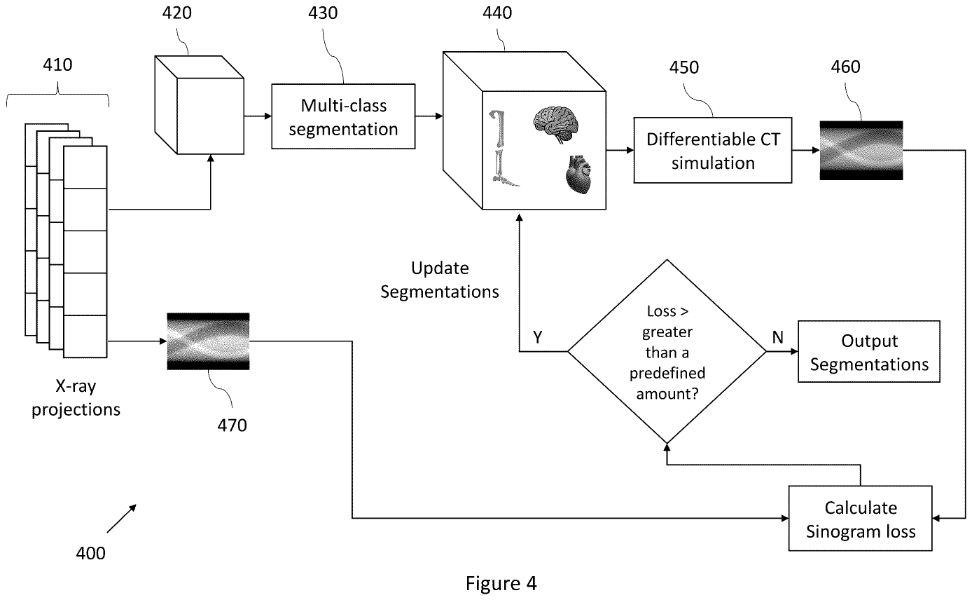

Reference is made to FIG. 4, which illustrates the overall flow of the process 400 performed by the system 310 when processing the X-ray projections 410.

As mentioned, the system 310 applies a reconstruction algorithm to the X-ray projections 410 to obtain an image 420. The image 420 in this case is a 3 dimensional (3D) image providing an image of a volume including at least part of the subject. The image 420 therefore comprises a plurality of voxels, where the intensity for each voxel represents the level of attenuation of the X-rays at the associated point in space. The reconstruction algorithm applied by the system 310 may, for example, be filtered backprojection.

In addition to providing the reconstructed image 420, the system 310 is also configured to process the X-ray projections to produce a sinogram 470. The sinogram 470 is a visual representation of the X-ray projections within the entire three dimensional (3D) imaging space. The sinogram 470 may be a two dimensional (2D) image. In the case that the detectors 345 are configured to integrate over the energy of different photons, each pixel in the sinogram 470 has a scalar value representing an attenuation value associated with the entire spectrum of energies. On the other hand, in the case that the detectors 345 are configured to measure the energies of different photons (e.g. in PCCT) to produce an energy spectrum, each position in the sinogram may be associated with a vector, wherein for each vector, each element in the respective vector is associated with a different range of energy values (i.e. each vector provides an energy spectrum). In this case, the sinograms 460, 470 may each be considered to be three dimensional (3D) sinograms, in which a first axis is associated with the angle of the detector 330, a second axis is associated with one of the detectors 345, and a third axis is associated with photon energies. The sinogram 470 is generated by the system 310 for the purposes of updating the segmentations determined based on the reconstructed image 420.

The system 310 applies a segmentation algorithm 430 to the reconstructed image 420 in order to determine the segmentations. Each segmentation may represent a separate structure within the subject 340. For example, the segmentations may include a first segmentation representing the heart or a part of the heart, and a second segmentation representing a first one of the lungs or a part of a first one of the lungs. The segmentations are determined based on differences in attenuation characteristics of the tissues belonging to the different structures. The segmentation algorithm 430 may comprise a machine learning model, e.g. a convolutional neural network (CNN), trained to determine the segmentations based on the image 420. The segmentation algorithm 430 may alternatively comprise a classical rule based segmentation algorithm.

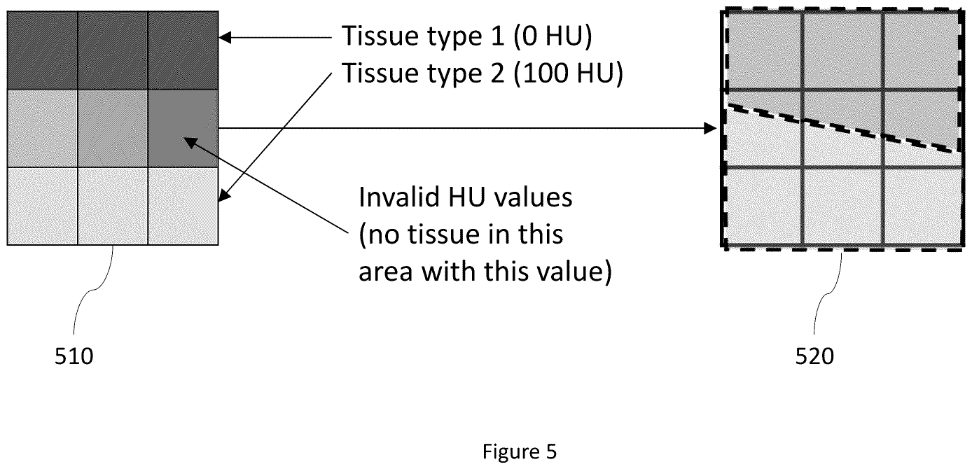

The determined segmentations may be provided as a mesh having a higher resolution than the image 420 itself. Reference is made to FIG. 5, which illustrates how part of the mesh between two segmentations may be determined based on voxels with a part 510 of the image 420. The image part 510 is a two dimensional (2D) cross-section of a small number of voxels belong to the 3D image 420. The image part 510 illustrates the boundary between two tissues, indicated as tissue type 1 and tissue type 2. The two different tissue types have different attenuation coefficients, which is reflected in the different Hounsfield units (HU), e.g. 0 HU for tissue type 1 and 100 HU for tissue type 2. Due to the limited resolution of the imaging apparatus and the resulting image, there are a number of voxels that cannot be associated exclusively with one tissue type or another. This may be referred to as the partial volume effect and may be the result of the boundary between the two types of tissue falling within the voxels. These voxels are shown in the image part 510 with intensities that are between the intensity values of tissue type 1 and tissue type 2. The computer system 310 may determine, on the basis that there is no tissue type having an attenuation coefficient (e.g. no tissue type with a Hounsfield unit between 0 HU and 100 HU, such as 50 HU) corresponding to the intermediate intensity, that the segmentation boundary falls within the voxels having the intermediate intensity. As shown in the image part 520 in which the mesh separating the two tissue types is overlaid, the mesh indicating the boundary between the two tissue types falls within these voxels having the intermediate intensity. Hence, the mesh may represent the segmentation boundaries with higher resolution than the resolution of the image 420 itself. Each of the determined segmentations are stored by the data processing system 310 as a mesh.

In embodiments, when determining the mesh boundaries falling within voxels as described, the 3D image 420 may contain a first set of voxels each having an intensity value substantially equal to a first intensity associated with a first tissue type (e.g. a tissue type of 0 HU); a second set of voxels each having an intensity value substantially equal to a second tissue type (e.g. a tissue type of 100 HU). The image 420 further comprises one or more additional voxels each having an intensity value (e.g. corresponding to 50 HU) between the intensity associated with the first tissue type and the intensity associated with the second tissue type. The intensity value of the one or more additional voxels is not associated with any tissue type. The system 310 is configured to: create a boundary of the segmentation mesh through the one or more additional voxels. This segmentation representation is robust to partial volume effects.

Referring again to FIG. 4, the system 310 provides the segmentation information 440 to a differentiable simulation 450. The differentiable simulation 450 is configured to model the processes that create artefacts explicitly during simulation. The differentiable simulation 450 may be trained or constructed by a system (e.g. system 150). The differentiable simulation 450 may provide a differentiable forward projection model for determining the X-ray projection data (in the form of a sinogram) based on the segmentations. In addition to the segmentations themselves, the differentiable simulation 450 may also determine the sinogram based on one or more of the geometry of the X-ray source 320 and detector 330, the motion of the X-ray source 320 and detector 330 in rotation around the subject 340, the spectra of X-ray interactions with the different tissue types represented in the segmentation information 440.

The segmentation information provided to the simulation 450 may comprise a volume comprising the plurality of segmentation meshes and an indication of characteristics of each of the segmentations. For example, the characteristics of each of the segmentations may comprise an indication of the tissue type and/or an indication of the attenuation characteristics. In addition to the segmentations and their characteristics, the system 310 may also provide as inputs to the differentiable simulation 450, information relating to the scanner, e.g. scanner geometry information and scan protocol information. The segmentation information is determined specifically for the particular subject 340, whereas at least some of the additional inputs, e.g. tissue properties, scanner geometry, scan protocol, may be static, i.e. be the same for multiple different subjects. The data processing system 310 may also provide as an input to the differentiable simulation 450, the image 420 determined earlier.

The differentiable simulation 450 is configured to output a simulated sinogram 460 based on the inputs (including at least the segmentation information 440) received earlier. The simulated sinogram 460 may be generated by performing forward projection based on the inputs to determine the X-ray projections, and hence the sinogram 460 that would be expected to arise based on imaging of part of the subject 340. The simulated sinogram 460 that is generated may correspond to a different cross-section through the subject 340. The simulated sinogram 460 is associated with the sinogram 470 generated directly from the X-ray measurements obtained using the X-ray imaging apparatus. The simulation models the physical process and the way the different tissue types interact with the radiation.

The simulation 450 is defined on the segmentations 440, which as discussed above are represented as a mesh, not an intensity volume. Therefore, the simulation 450 will allow the determination of a more accurate boundary between tissue types than would be the case if the segmentations 440 were defined on a voxel level.

The data processing system 310 is configured to perform a comparison between a sinogram 470 and a corresponding simulated sinogram 460. A comparison is made between the sinogram 470 and the corresponding simulated sinogram 460 to determine a loss associated with the simulated sinogram 460. Each of the sinogram 470 and the corresponding simulated sinogram 460 may take the form of matrix of values. Each matrix may be a 2D matrix, in the case that the CT is an energy-integrating CT, or may be a 3D matrix, in the case that the CT is a photon-counting computed tomography (PCCT). Determining the loss may comprise calculating a loss metric based on an elementwise functions between the matrix representing the simulated sinogram 460 and the matrix representing the sinogram 470. The loss metric may, for example, be a mean squared error, a structural similarity index measure, or a normalized correlation. The loss metric may be differentiable, which allows the system 310 to calculate gradients based on the loss metric and to thereby perform back-propagation to update the segmentations 440. In some embodiments, the system 310 may not calculate gradients by using derivative information—e.g. in the case that one or more components are not differentiable—then the system may instead approximate the gradients for updating the segmentations, e.g. by performing a finite element approximation of the gradients.

Since the simulated sinogram 460 is a predicted sinogram based on the segmentation information, the loss associated with the simulated sinogram 460 is indicative of the error associated with a relevant part of the segmentations. If this loss is greater than a predefined amount, the loss is used by the data processing system 310 to update the segmentations 440. In some embodiments, the data processing system 310 may apply differentiation to guide segmentation updates so that the simulated and generated sinograms 460, 470 start to converge. In some embodiments, the data processing system 310 uses generative machine learning models to update the segmentations 440 based on the sinogram loss. In this case, the determined sinogram loss takes the form an AI loss term, which helps to avoid adversarial-attack-like problems. The AI loss term may take the form of an adversarial (GAN) loss, variational autoencoder (VAE) loss, perceptual loss, or cycle consistency loss.

Once the updated segmentations 440 have been determined, the system 310 provides these to the differentiable simulation 450, which is configured to produce a further simulated sinogram 460. The system 310 performs a comparison between the further simulated sinogram 460 and the corresponding sinogram 470 determined from the X-ray measurements to again determine a sinogram loss, which if greater than the predefined amount, is used to update the segmentations 440. The process may continue by iteratively updating the segmentations 440 and comparing sinograms 460, 470 in this manner until the loss is less than a predefined amount. Once the calculated loss is less than the predefined amount, the system 310 outputs the segmentations resulting from the most recent update as the result. In some embodiments, the process of iteratively updating the segmentations 440 and comparing sinograms 460, 470 may continue until the loss has not reduced by a predefined threshold for a predefined number of iterations. This may be referred to as a convergence criterion for determining when to output the segmentations. The convergence criterion may be applied by the system 310 in addition to the criterion of the predefined threshold—i.e. in case either of the two criteria (the loss being less than the predefined amount or the loss not changing by the predefined threshold for a predefined number of iterations) are met the segmentations may be output.

In some embodiments, the updated segmentation meshes may comprises parts provided at different resolutions. The resolution of the segmentation meshes may be determined by the system 310 in dependence upon the initial segmentation results and the gradients determined based on the sinogram loss. For example, the resolution of the segmentation meshes may be set to be greater where the system 310 determines that there is a high level of curvature in the initial segmentation results. The use of lower resolution for parts of the segmentation meshes enables the process of updating the segmentation meshes to be performed more quickly.

In some embodiments, the differentiable simulation 450 may be configured to model time and to output the simulated sinogram 460 based on a time component. If time is not considered when the image 420 is reconstructed and the segmentations 440 determined, the motion of the subject 340 may result in blurring in the areas of motion represented in the image 420 and hence inaccuracies in the segmentations 440.

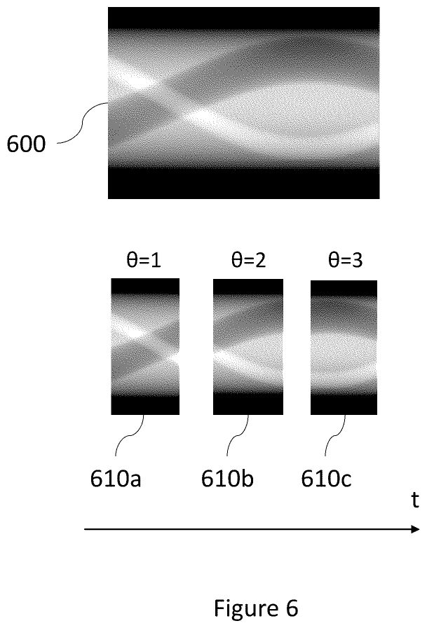

The sinogram capture information collected at different times. Reference is made to FIG. 6, which illustrates an example sinogram 600. The sinogram 600 may be the sinogram 470 constructed directly from the X-ray measurements 410 by the system 310. One of the axes (i.e. the horizontal axis in FIG. 6) of the sinogram 600 represents the angle at which the corresponding X-ray projection data is obtained. Different slices 610a-c of the sinogram 600 are associated with different angles (θ=1, θ=2, θ=3) at which the X-ray measurement/s corresponding to the respective slice are collected. Since the X-ray measurements corresponding to the different slices 610a-c are captured as the X-ray source 320 and detector apparatus 330 rotate around the subject 340, the X-ray measurements captured at different angles are also captured at different times. In FIG. 6, it is shown that the slice 610c represents X-ray measurement data collected at a third angle (θ=3) at a later time than the X-ray measurement data collected at a second angle (θ=2). The X-ray measurement data collected at the second angle (θ=2) is collected at a later time than the X-ray measurement data collected at the first angle (θ=1). Different positions along the vertical axis of the sinogram 600 may be associated with different ones of the detectors 345.

Referring back to FIG. 4, the sinogram 470, therefore, contains X-ray measurement data collected over a period of time as the X-ray source 320 and detection apparatus 330 rotate around the subject 340. The time information may be accounted for by the system 310 when updating the segmentations 440 in order to recover the movement, e.g. by providing a plurality of frames of segmentation data representing changes to the position of different structures of the subject 340 over time. Alternatively, the time information may be accounted for by the system 310 when updating the segmentations 440 in order to remove motion artefacts from the 3D volume containing the segmentations. To achieve this, a number of frames of the segmentation information 440 are generated, the simulation 450 may use of each of these frames along with time information indicating when the respective frame was generated.

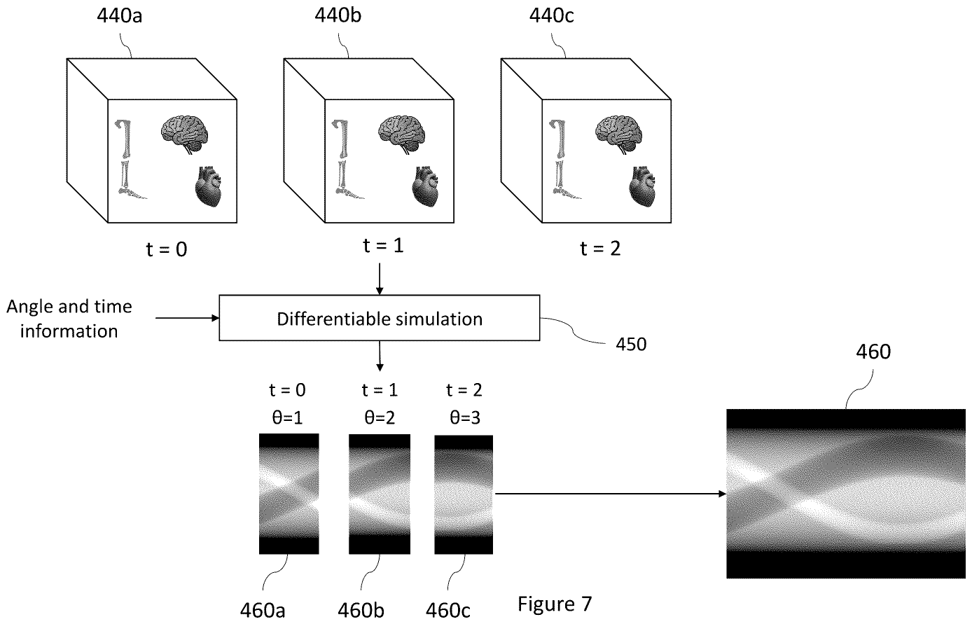

Reference is made to FIG. 7, which illustrates how a number of frames 440a to 440c of the segmentation information 440 may be processed by the simulation 450 to produce the sinogram 460. Each of the frames 440a to 440c represents the segmentation of structures within the subject 340 at a different time. Each of the frames 440a to 440c is received by the differentiable simulation 450, which additionally receives angle and time information indicating the times at which the X-ray source 320 and detector apparatus 330 are arranged at a particular angle. Each of the frames 440a to 440c may be matched to a particular angle of X-ray measurement/s. Based on the angle, time, and frames 440a to 440c of segmentation information, the differentiable simulation 450 outputs each of the slices 460a to 460c of the sinogram 460, where each of the slices 460a to 460c corresponds to one of the frames 440a to 440c. When the loss between the simulated sinogram 460 and the sinogram 470 is determined, the determined loss term may be associated with all of the angles (and therefore times). The loss associated with each of the angles may be used to update each of the frames 440a to 440c associated with that angle.

In some embodiments, once the system 310 determines that the calculated loss associated with the simulated sinogram 460 produced based on the frames of segmentation data 440a to 440c is less than the predefined amount, the system 310 outputs the frames of segmentation data 440a to 440c used to produce the simulated sinogram 460. In other embodiments, once the system 310 determines that that the sinogram loss is less than the predefined amount, the system 310 may output a single frame of segmentation information in which the motion artefacts are corrected for.

The update of the segmentations 440 may be performed by the system 310 using a generative neural network, which receives data representing the segmentations 440 as an input and the sinogram loss and conditioning information. Such a generative neural network may include a generative adversarial network (GAN), a diffusion network, or a transformer.

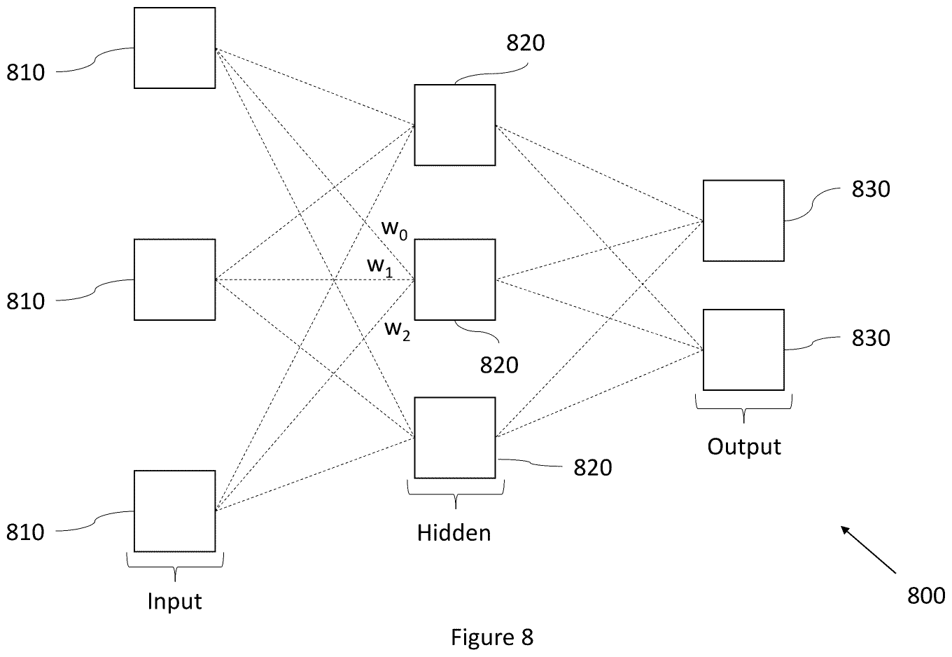

FIG. 8 as a schematic illustration of a neural network 800. The neural network 800 comprises input nodes 810, hidden nodes 820 and output nodes 830. In practice, there are likely to be many more nodes in the network 800 than those shown, and more hidden layers than the one shown. Each input node 810 receives a single value of the input data and produces at its output, an activation or node value, which is generated by supplying the input value to an activation function (e.g. a sigmoid). Each of the input nodes 810 is connected to each of the hidden nodes 820. A matrix of weights defines the connectivity between the input nodes 810 and the hidden nodes 820. A vector of the node values output from the input nodes 810 is scaled by a vector of respective weights at the input of each of the hidden nodes 820, each weight defining the connectivity of one of the input nodes 810 with a connected one of the hidden nodes 820. The weights applied at the inputs of one of the hidden nodes 820 are shown in FIG. 8 as w₀ . . . w₃. At each hidden node 820, the input value at that node is given by the dot product of its associated weights vector and the output values of the input nodes 810. The activation function is then applied to the input values at the hidden nodes 820 to provide the output values of those nodes 820. The output vector of the hidden nodes 820 is supplied to each of the nodes 830 in the next layer of the network 800 and used in a similar manner to generate the output values for that next layer.

The network 800 may be trained through supervised or unsupervised learning. In one embodiment, the network 800 is trained through supervised leaning by determining at least one set of output values based on at least one set of input values included in the training data. The output values are compared to known labels in the training data and an error or loss is calculated (i.e. based on a difference between the output values and the labels). The error or loss is then back-propagated through the network 800 to update the weights, such that the network 800 is trained to better approximate the labels from the input values. In the next cycle, the revised weights are used with further training data to further update the weights to more closely reproduce the labels of the further training data based on the input values of the further training data. In this way, the network 800 can be trained to perform a specific task.

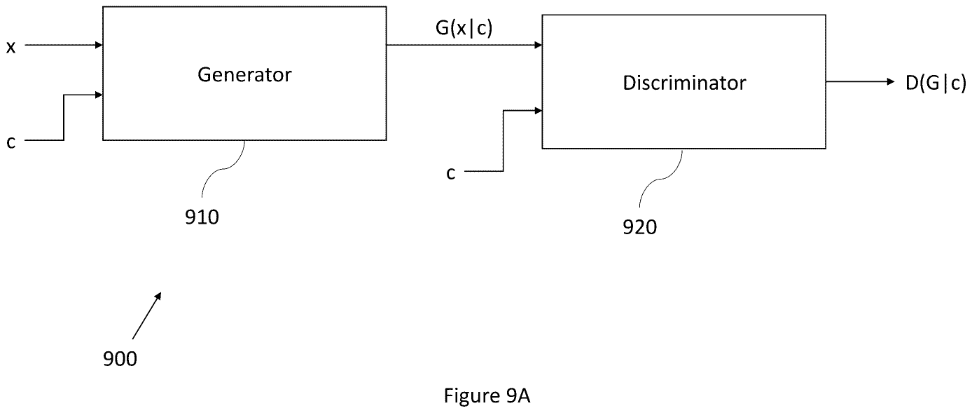

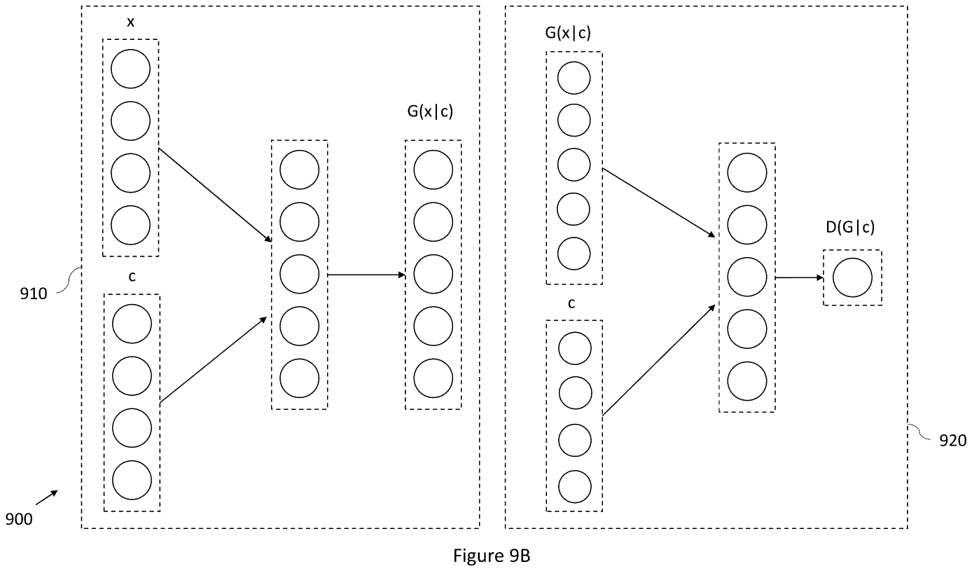

In some embodiments, the image transformation model 235 may comprise a generator model of a generative adversarial network (GAN). Reference is made to FIGS. 9A and 9B, which illustrates an example of a GAN 900.

FIG. 9A illustrates two components of a GAN 900. A first component 910 is referred to as the generator 910 and is configured to generate outputs G(x|c) based upon a particular input, shown as x. The input x in the present case is the segmentation information, which is to be subject to updating based on the calculated sinogram loss. The generator 910 may also receive condition information, shown as c, which is provided as an additional input layer into the generator 910. The condition information in the present case is the sinogram loss. The generator 910 produces an output, G(x|c), which depends on both the input x and the condition information c. When the generator 910 has been trained and used for inference by the system 310, the output G(x|c) represents updated segmentation information.

The GAN 900 further includes a second component, referred to as the discriminator 920, which is used as part of the training process for training the generator 910. The discriminator 920 is trained to provide scores for the volume data G(x|c) output by the generator 910, where the scores indicate how closely the generated segmentations G(x|c) align with a set of training segmentations. In other words, the discriminator 920 is trained to identify whether segmentations reflect a real segmentations or a generated segmentations. The discriminator 920 receives as an input, the data G(x|c) output by the generator 910, which represents input segmentations. The discriminator 920 also receives the same condition information, c, as received by the generator 910 at an additional input layer at the discriminator 920.

The generator 910 and the discriminator 920 are trained as part of a same training process in which the loss function of the discriminator 920 is used to update the model parameters of both the generator 910 and the discriminator 920. This training process may be performed by computing system 150. The generator 910 produced by the training process may be provided by the system 310 for updating the segmentations 440. The generator 910 may also receive as condition information, data representing the sinogram loss.

FIG. 9B illustrates a simplified example of a generator 910 and a discriminator 920 in which certain example layers are illustrated. As shown, the generator 910 receives an input, x, which represents input segmentations to be updated by the generator 910. The generator 910 also receives condition information, c. Both x and c are mapped to hidden layers of the neural network of the generator 910 with a given activation function. In response to these inputs, the generator 910 produces an output, G(x|c), which represents output segmentations.

The discriminator 920 receives an input, G(x|c), which represents the segmentation information determined by the system 310. The discriminator 920 also receives condition information, c, which may include the sinogram loss determined by the system 310. The conditioning information, c, may further include gradients derived based on the sinogram loss and determined by the system 310. Both x and c are mapped to hidden layers of the neural network of the discriminator 920 with a given activation function. In response to these inputs, the discriminator 920 produces an output, D(G|c), which represents a score for the input G(x|c).

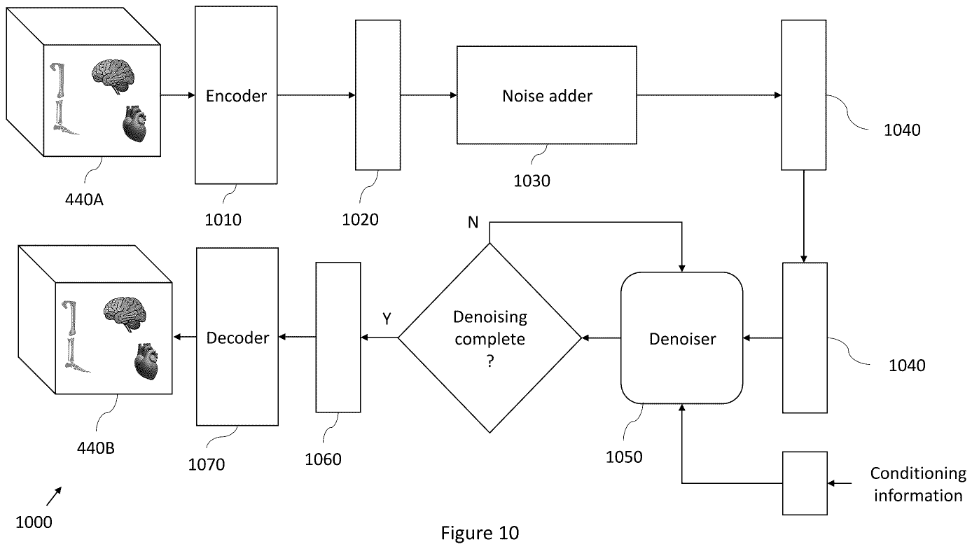

Reference is made to FIG. 10, which illustrates how a diffusion model may operate to transform an input image (segmentation volume 440A in this example) into an output image (an updated version of the segmentation volume 440B in this example). The diffusion model 1000 comprises an encoder 1010, which is configured to encode the segmentation volume 440A to transform the image from pixel space to a latent space. The image 1020 output from the encoder 1010 represents the input volume 440A in latent space. A diffusion model 1000 comprises a module 1030, which is configured to add noise to the input volume 440A to generate a noisy image 1040. The noisy image 1040 retains some of the information from the original volume 440A. The noisy image 1040 is input to a denoiser module 1050 of the image transformation model 1000. The denoiser module 1050 comprises a CNN that is trained to iteratively remove noise from the image 1040. The image denoising is performed over a number of iterations until the output image 1060 is produced. The output image 1060 is provided to the decoder 1070, which is configured to convert the image 1060 into pixel space to produce output volume 440B, which represents the updated segmentation information.

Additionally, conditioning information may be applied to the denoising process to control the transformation process. The conditioning information is supplied to an encoder 1080, which converts the condition information into a set of values suitable for applying to the denoiser 1050 via a cross-attention mechanism. The conditioning information may comprise loss term or data (e.g. gradients) derived based on the loss term. The conditioning information may be applied via a cross-attention mechanism to the denoiser 1050.

The diffusion model 1000 may be trained to create output images (i.e. updated segmentations) in an adversarial manner by training a discriminator 620 to assign quality scores to the output images of the diffusion model 1000, indicating the extent to which the output images correspond to desired outputs.

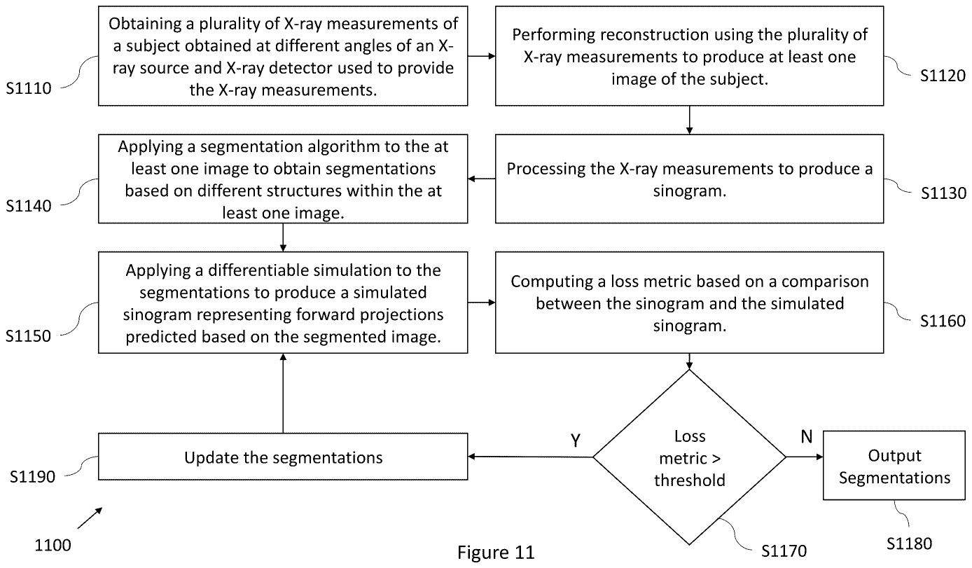

Reference is made to FIG. 11, which illustrates an example method 1100 according to example embodiments. The method 1100 may be performed in response to at least one processor of the data processing system 100 executing computer readable instructions belonging to a computer program. The computer program may be stored on a non-transitory computer readable medium.

At S1110, the system 310 obtains a plurality of X-ray measurements of a subject obtained at different angles of an X-ray source 320 and X-ray detector 330 used to provide the X-ray measurements. The system 310 may obtain these measurements from the X-ray imaging apparatus comprising the X-ray source 320 and X-ray detector 330.

At S1120, the system 310 performs reconstruction using the plurality of X-ray measurements to produce at least one image of the subject.

At S1130, the system 310 processes the plurality of X-ray measurements to produce a sinogram (e.g. sinogram 470). Although shown as being performed after S1120, S1130 may be performed prior to or simultaneously with S1120.

At S1140, the system 310 applies the segmentation algorithm 430 to the at least one image 420 to obtain segmentations 440 based on different structures within the image.

At S1150, the system 310 applies the differentiable simulation 450 to the segmentations 440 to produce a simulated sinogram 460 representing forward projections predicted based on the segmented image 440.

At S1160, the system 310 computes a loss metric based on a comparison between the sinogram 460 and the simulated sinogram 470.

At S1170, the system 310 determines whether the less metric is greater than a threshold. If not, at S1180, the system 310 outputs the segmentations 440, e.g. by storing them to a memory as the result of the method 1100 for determining the segmentations or by outputting them to a display. Alternatively, if the segmentations are greater than the threshold, at S1190, the segmentations are updated based on the calculated loss metric. After S1190, the method 1100 proceeds to S1150 at which the system 1100 uses the updated segmentations to produce another simulated sinogram 460 representing forward projections predicted based on the updated segmentations. The system 310 continues to perform iterations of S1150, S1160, and S1190, until the loss metric is determined at S1170 to no longer be greater than the threshold.

Implementations of the subject matter and the operations described in this specification can be realized in digital electronic circuitry, or in computer software, firmware, or hardware, including the structures disclosed in this specification and their structural equivalents, or in combinations of one or more of them. For instance, hardware may include processors, microprocessors, electronic circuitry, electronic components, integrated circuits, etc. Implementations of the subject matter described in this specification can be realized using one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

According to various embodiments, there is provided: a CT scanning machine which produces the actual sinogram; an initial reconstruction step which converts the actual sinogram into an intensity volume and a subsequent multi-class segmentation algorithm which produces initial segmentations; differentiable simulation of the scanning process which uses the initial segmentations to produce a simulated sinogram; a loss function which computes a loss metric between the actual sinogram and simulated sinogram; an optimization loop which iteratively updates the segmentations according to gradients calculated from the loss metric. In some embodiments, the initial segmentations are used as a starting point for distinct sets of segmentations for each time point. In some embodiments, the initial segmentations and simulation use a representation that is higher resolution than the intensity volume: higher resolution voxel map, surface distance, mesh. In some embodiments, the initial segmentations are used to determine regions of interest that use higher resolution representations and the other areas use lower resolution representations. In some embodiments, the differences between the segmentations at each time point are used to estimate motion. In some embodiments, the optimization loop uses a generative model to update the segmentations. In some embodiments, where the loss function is a learned function trained to maximise an objective, in particular a perceptual objective. In some embodiments, a PCCT scanner is used and the simulation is modified to account for the different physics involved, for example the way different tissue types respond to different frequencies and contrast agents.

While certain arrangements have been described, the arrangements have been presented by way of example only, and are not intended to limit the scope of protection. The inventive concepts described herein may be implemented in a variety of other forms. In addition, various omissions, substitutions and changes to the specific implementations described herein may be made without departing from the scope of protection defined in the following claims.

1. A data processing system comprising processing circuitry configured to:
obtain a plurality of X-ray measurements of a subject obtained at different angles of an X-ray source and X-ray detector used to provide the X-ray measurements;
process the X-ray measurements to produce a sinogram;
perform reconstruction using the plurality of X-ray measurements to produce at least one image of the subject;
apply a segmentation algorithm to the at least one image to obtain segmentations based on different structures within the at least one image;
apply a differentiable simulation to the segmentations to produce a simulated sinogram representing forward projections predicted based on the segmentations;
compute a loss metric based on a comparison between the sinogram and the simulated sinogram; and
update the segmentations based on the loss metric.

2. The data processing system as claimed in claim 1, wherein the processing circuitry is configured to update the segmentations by providing the segmentations and loss metric to a generative machine learning model trained to output updated segmentations.

3. The data processing system as claimed in claim 2, wherein the processing circuitry is configured to input the loss metric to the generative machine learning model as conditioning information.

4. The data processing system as claimed in claim 2, wherein the generative machine learning model comprises at least one of:
a generative adversarial network (GAN);
a diffusion model; and
a transformer.

5. The data processing system as claimed in claim 1, wherein the plurality of X-ray measurements are photon-counting computed tomography (PCCT) measurements, each of which comprises energy spectrum data.

6. The data processing system as claimed in claim 1, wherein the segmentations comprise a plurality of segmentations meshes.

7. The data processing system as claimed in claim 6, wherein for each of least one the segmentation meshes, the resolution varies between different parts of the respective segmentation mesh.

8. The data processing system as claimed in claim 7, wherein the processing circuitry is configured to for each of at least one of the segmentation meshes, vary the resolution of the respective segmentation mesh in dependence upon at least one of:
gradients determined based on the loss metric; and
a level of curvature in initial segmentation results for the segmentations derived based on the segmentation algorithm.

9. The data processing system as claimed in claim 6, wherein the image of the subject comprises:
a first set of voxels each having an intensity value substantially equal to a first intensity associated with a first tissue type;
a second set of voxels each having an intensity value substantially equal to a second intensity associated with a second tissue type; and
one or more additional voxels each having an intensity value between the first intensity associated with the first tissue type and the second intensity associated with the second tissue type,
wherein the processing circuitry is configured to create a boundary of the segmentation mesh through the one or more additional voxels.

10. The data processing system as claimed in claim 1, wherein the at least one image comprises a plurality of image frames each associated with a different one of a plurality of time points, wherein the segmentations comprise a plurality of sets of segmentation data, wherein each of the sets is associated with a different one of the plurality of time points, wherein the processing circuitry is configured to perform at least one of:
output the plurality of sets of segmentation data showing movement of the different structures within the at least one image over time; and/or output a further set of segmentation data based on the plurality of sets of segmentation data in which the movement of the different structures is corrected for.

11. The data processing system as claimed in claim 1, wherein the processing circuitry is configured to:
determine whether the computed loss metric exceeds a predefined threshold; and
perform the update of the segmentations in response to determining that the computed loss metric exceeds the predefined threshold.

12. The data processing system as claimed in claim 11, wherein the processing circuitry is configured to:
until the computed loss metric no longer exceeds the predefined threshold, iteratively perform steps of updating the segmentations based on the loss metric, applying the differentiable simulation to the updated segmentations to produce an updated simulated sinogram and computing the loss metric using the updated simulated sinogram; and
in response to determining that the computed loss metric no longer exceeds the predefined threshold, outputting the segmentations resulting from a most recent iteration of the steps.

13. The data processing system as claimed in claim 12, wherein outputting the segmentations comprises at least one of:
storing the segmentations resulting from the most recent iteration as a result in memory;
outputting the segmentations resulting from the most recent iteration over a network to a further system; and
controlling a display to show a visual representation of the segmentations resulting from the most recent iteration.

14. A computer implemented method comprising:
obtaining a plurality of X-ray measurements of a subject obtained at different angles of an X-ray source and X-ray detector used to provide the X-ray measurements;
processing the X-ray measurements to produce a sinogram;
performing reconstruction using the plurality of X-ray measurements to produce at least one image of the subject;
applying a segmentation algorithm to the at least one image to obtain segmentations based on different structures within the at least one image;
applying a differentiable simulation to the segmentations to produce a simulated sinogram representing forward projections predicted based on the segmentations; and
computing a loss metric based on a comparison between the sinogram and the simulated sinogram; and
updating the segmentations based on the loss metric.

15. A computer program comprising one or more sets of computer-readable instructions stored on a non-transitory computer-readable medium, which when executed by at least one processor, cause a method to be performed, the method comprising:
obtaining a plurality of X-ray measurements of a subject obtained at different angles of an X-ray source and X-ray detector used to provide the X-ray measurements;
processing the X-ray measurements to produce a sinogram;
performing reconstruction using the plurality of X-ray measurements to produce at least one image of the subject;
applying a segmentation algorithm to the at least one image to obtain segmentations based on different structures within the at least one image;
applying a differentiable simulation to the segmentations to produce a simulated sinogram representing forward projections predicted based on the segmentations; and
computing a loss metric based on a comparison between the sinogram and the simulated sinogram; and
updating the segmentations based on the loss metric.
